(19) United States
(12) Patent Application Publication
(10) Pub. No.: US 2008/0280995 A1
(43) Pub. Date: Nov. 13, 2008

(54) Extracts of Latex of Calotropis Procera and to a Method of Preparation Thereof

(75) Inventors: Vijay L. Kumar, New Delhi (IN); Vijay Kumar, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignees: ALL INDIA INSTITUTE OF MEDICAL SCIENCES, NEW DELHI (IN); INTERNATIONAL CENTRE FOR GENETIC ENGINEERING AND BIOTECHNOLOGY, New Delhi (IN)

(21) Appl. No.: 11/578,632

(22) PCT Filed: Apr. 7, 2005
(86) PCT No.: PCT/IN05/00106

§ 371 Date: Jul. 25, 2007

(30) Foreign Application Priority Data
Apr. 10, 2004 (IN) 737/DEL/2004

(51) Int. Cl. A61K 31/045; A61P 35/00

(52) U.S. Cl. 514/724

(57) ABSTRACT

The invention relates to aqueous suspension of dried latex (DL) of Calotropis procera and to a method of preparation for the prevention/treatment of cancer. The DL suspension when administered orally in the X-myc mice—a transgenic mouse model of hepatocellular carcinoma (HCC), was effective in protecting the animals from the atypical mitosis and displastic/neoplastic changes occurring in vivo. Further, the DL suspension did not show any observable side effects when administered orally to the animals for 16 weeks. Further, the purified fractions of the latex were found to exhibit potent cytotoxic activity in in vitro cell culture system using two different cancer cell lines.

### FIELD OF INVENTION

This invention relates to extracts of latex of Calotropis procera and to a method of preparation that can be advantageously employed.

### PRIOR ART

The incidence of cancer is increasing worldwide and it is the single most common cause of death in developed and developing countries. It results from an uncontrolled growth of cells that proliferate and exhibit atypical mitosis and dysplasia/neoplasia. Through extensive research in the past five decades, several causative agents and risk factors of cancer have been identified and their molecular mechanisms worked out in detail. These include smoking, tobacco chewing, alcohol consumption, hormonal imbalance and chronic disease like hepatitis. Besides, genetic factors also predispose an individual to cancer. The expression of a number of oncogenes has been associated with the occurrence of cancer. Deregulated expression of c-myc is often associated with poor prognosis. The survival rate of patients in most of the cancers is poor. Surgical removal of localized tumors has given best results. However, once metastasized (spread to other body parts), cancer cannot be removed and the treatment relies mainly on the drug therapy, i.e. chemotherapy and radiation therapy. Recent epidemiological studies have shown that prolonged use of aspirin or other non-steroidal drugs reduces the risk of colon cancer by 40-50%. Non-steroidal anti-inflammatory drugs NSAIDs) also inhibit chemically induced colon carcinoma in animal model. A number of herbal preparations have also been found effective against different cancers.

In the traditional Indian medicinal system, the Ak plant or Calotropis procera—has been used for a variety of disease conditions like leprosy, ulcers, tumors, piles and diseases of spleen, liver and abdomen. The root extract of Calotropis procera has been shown to display strong cytotoxic effect in COLO 320 tumor cells [Smit H F, Woerdenbag H J, Singh R H, Meulenbeld G J, Labadie R P, Zwaving J H. (1995) Ayurvedic herbal drugs with possible cytostatic activity. J Ethnopharmacol. 47: 75-84]. However, the in vivo anti-tumor activity of Calotropis extracts have not been investigated.

### Medicinal And Toxic Properties of Calotropis Procera

Calotropis procera is a wild growing plant that belongs to the family Asclepiadaceae. It is known by various names like swallow wort, dead sea apple, sodom apple or milk weed. In India the plant is known by different names like Ak in hindi, orka in Oriya, Alkarka in Sanskrit and Vellerukku in Tamil. In the traditional Indian medicinal system, it has been used for a variety of disease conditions like leprosy, ulcers, tumors, piles and diseases of spleen, liver and abdomen. The plant is also known for its toxic properties that include iridocyclitis, dermatitis and acts like a poison and produces lethal effects. The aqueous extract of the dried latex produces inflammation when administered by subcutaneous injection.

Leaves and roots of this plant have been used to relieve pain under different conditions [The Wealth of India (1992) Raw Materials, Vol. 3, pp 78-84]. The aqueous extract of dry latex of this plant also acts as an analgesic and antipyretic [Dewan S., Sangraula H. and Kumar V. L. (2000) Preliminary studies on the analgesic activity of latex of Calotropis procera. J Ethnopharmacol, 73: 307-311; Dewan S., Kumar S. and Kumar V. L. (2000) Antipyretic effect of latex of Calotropis procera. Ind. J. Pharmacol. 32:252]. The ethanol extracts of its flowers [Mascolo N., Sharma R., Jain S. C. and Caspasso F. (1988) Ethnopharmacology of Calotropis procera flowers. J Ethnopharmacol. 22.211-221], and aqueous extracts of dry latex of this plant exhibit strong anti-inflammatory activity in rat paw edema model [Kumar V L, Basu N. (1994) Anti-inflammatory activity of the latex of Calotropis procera. J. Ethnopharmacol. 44: 123-125]. The chloroform soluble fraction of its root exhibits anti-inflammatory activity in formaldehyde-induced arthritis model [Basu A. and Nag Choudhuri A. K. (1991) Preliminary studies on the anti-inflammatory and analgesic activities of Calotropis procera root et. J Ethnopharmacol. 31: 319-324]. Decoction of the aerial parts of this plant exhibits antipyretic, analgesic and neuromuscular blocking activity [Mossa J. S., Tariq M., Mohin A., Ageel A. M., al-Yahya M. A., al-Said M. S. and Rafatullah S. (1991) Pharmacological studies on aerial parts of Calotropis procera. Am J Chin Med 19:223-231].

Patent (W003055558) describes the use of a polyherbal composition containing Calotropis procera root for the treatment of bronchial asthma.

Patent (GB398547) describes the use of Calotropis procera flax for the improvement of acoustic plaster.

Although a number of drugs are available for the management of cancer none of the drugs is safe when used over a long period of time. Treatment with some of the drugs even results in resistance to therapy. A number of compounds have been shown to possess anticancer activity but most of them produce serious side effects. Even NSAIDs produce gastric and renal side effects on long-term use and alter platelet function. COX-2 selective inhibitors also produce gastric and renal side effects on long-term use.

As a result, a number of plant-derived substances have been made available through intensive research [Wargovich M J, Woods C, Hollis D M, Zander M E. (2001) Herbals, cancer prevention and health. J Nutr. 131 (11 Suppl): 3034S-6S]. The present invention is plant-derived extract/product obtained from the latex that is orally effective as an anticancer agent in a transgenic mouse model of cancer. It is free from observable side effects when used for 16 weeks. The animals were protected form the dysplasic changes occurring due to the expression of an oncogene. Further, the purified fractions of the latex were found to exhibit potent cytotoxic activity in in vitro cell culture system using two different cancer cell lines.

### OBJECTS OF THE INVENTION

An object of this invention is to propose a plant-derived product for the treatment and prevention of cancer that is free from side effects on long-term use.

Another object of this invention is to propose an aqueous extract of dried latex of Calotropis procera for the treatment and prevention of cancer.

### DESCRIPTION OF INVENTION

The present invention relates to an aqueous extract of dried latex (DL) of Calotropis procera for the treatment and prevention of cancer. The extract can only be given orally and is free from observable side effects. The fractions prepared from the dried latex also exhibit cytotoxic activity in cancer cell lines.

According to this invention there is provided an extract of latex of Calotropis procera for use as cytotoxic and anticancer agents leaving as identified by preparation by thin layer chromatography.

Further, according to this invention there is provided a process for preparing extracts of latex of Calotropis procera for use as cytotoxic and anticancer agents comprising in the step of subjecting a methanol extract of dried latex of Calotropis procera to the steps of emotion with a non-polar solvent followed by a polar solvent.

### DETAILED DESCRIPTION

The latex of Calotropis procera contains the active ingredients for use in the treatment and prevention of cancer. The latex is collected from the aerial parts of the plant growing in the wild that include flower, bud and leaves. It is dried under shade to obtain a solid material that is then kept at room temperature for about two to three months (dried latex i.e., DL). The small-scale extraction involves triturating a small amount of DL e.g., 50 milligrams (mg) to 100 mg in 1 to 2 milliliter of water using a pestle and mortar. The aqueous suspension or crude extract thus obtained contains the active constituents but not yet characterized or identified.

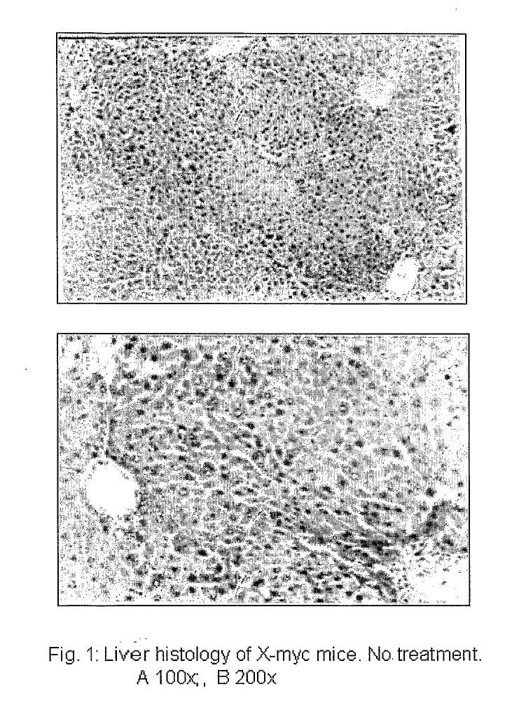

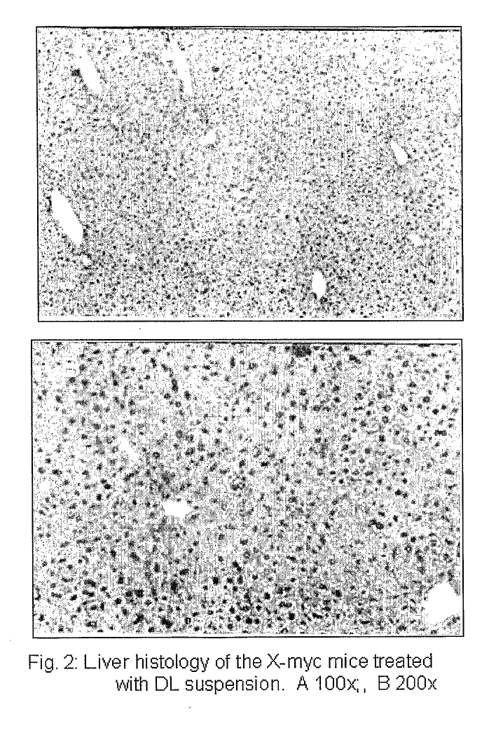

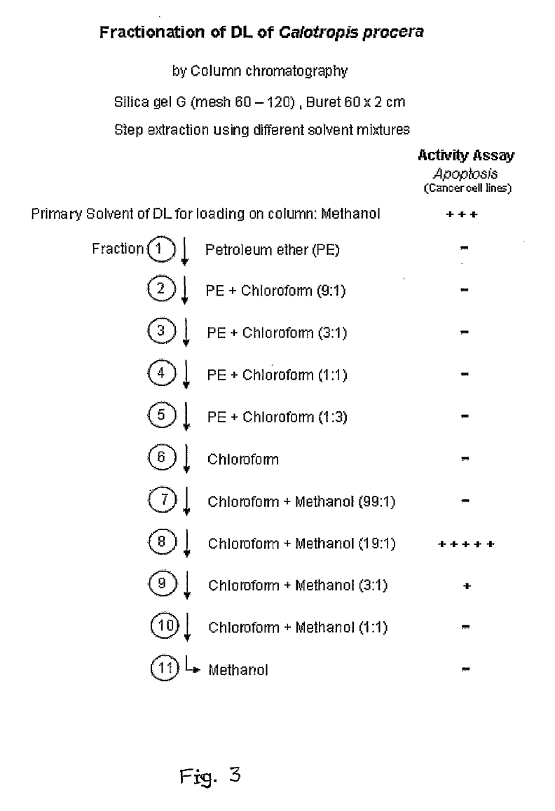

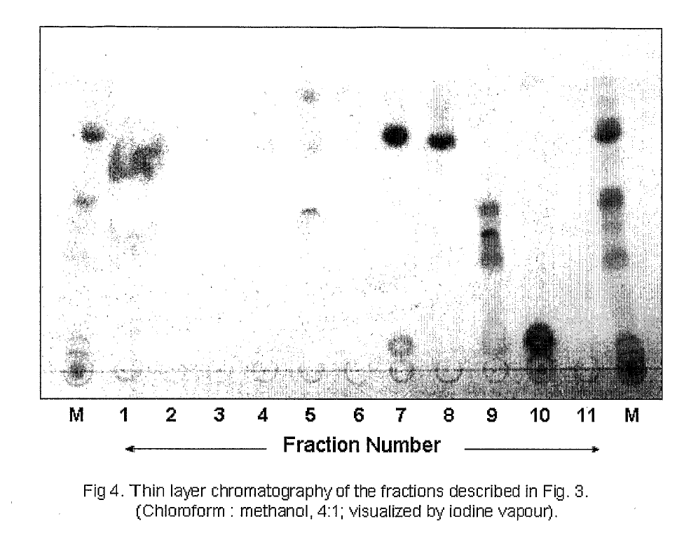

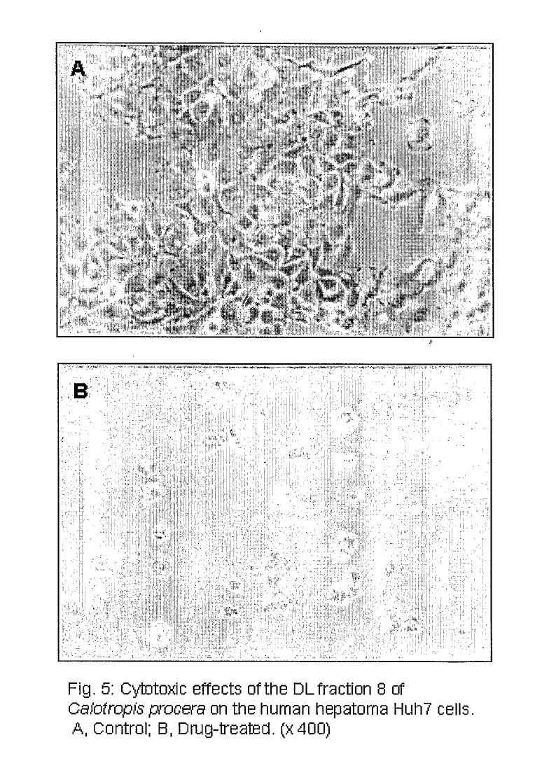

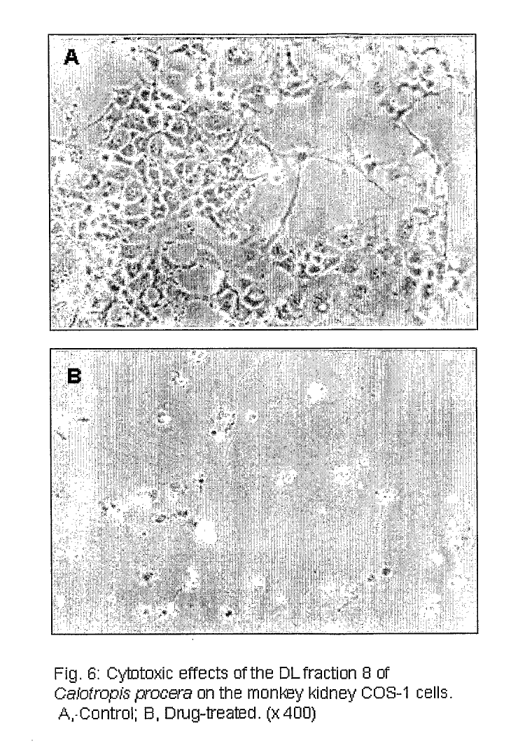

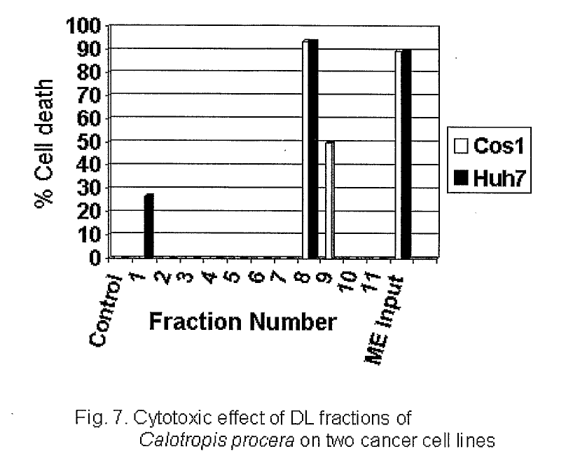

The DL was fractionated by chromatography and the fractions thus obtained were tested for cytotoxic activity in two cancer cell lines. Example 1 Transgenic mice expressing myc oncogene in the liver and thereby resulting in hepatocellular carcinoma (patent No. U.S. Pat. No. 6,274,788) were used for this study. These mice exhibit atypical mitosis and dysplasia in the liver on histological examination as early as 10-12 weeks of age. The overnight fasted animals were fed with bread soaked in aqueous suspension of the DL at a does of 400 mg (5 days/week) (3 male and 3 female mice). The control animals were given bread alone (4 female mice). The treatment started when the animal were 5 weeks old and was continued till the animals were 20 weeks old when the animals were sacrificed and their livers were dissected out and preserved in 10% neutral buffered formalin. Sections were prepared and stained with eosin and hemotoxylin and examined under microscope. The results (FIG. 1) show atypical mitosis and dysplasia in the livers of control animals. FIG. 2 shows the effect of treatment with aqueous extract of DL where the liver appears to be normal. Example 2 the steps of fractionation are illustrated in FIG. 3 of the accompanying drawings it being understood that the numerical values illumed in FIG. 3 are only by way of example. The DL was soxhlated with petroleum ether (B.P. 40-60° C.), methanol in a sequential order. The methanol extract was subjected to column chromatography using Silica Gel G (mesh 60-120) in a column (60×2 cm). The sample was loaded in methanol and the elation was carried out with solvent start form non-polar solvent to polar solvents as given in FIG. 3. The eluted fractions were analyzed by thin layer chromatography using silica gel G plates and chloroform:methanol (4:1) as mobile phase and iodine was used to visualize the bands. FIG. 4 shows the thin layer chromatogram of these Nations. The solvent was vaporated and the dry fractions were stored at room temperature in desiccator. They were dissolved in methanol and further diluted with aqueous vehicle for testing the cytotoxic activity. Example 3 the fractions obtained in the example 2 were tested for cytotoxic activity in two cancer cell lines i.e. COS cells (ATCC CRL 1650) and Huh-7 cells [Nakbayashi H., Taketa K., Miyano K., Yamane T. and Sato J. (1982) Growth of human hepatoma cells lines with differentiated functions in chemically defined medium. Cancer Res. 42: 3858-3863]. The cells (5×10⁵ cells) were plated in 3 ml DMEM with 10% fetal bovine serum in 60 mm dishes. The fractions were added after dilution with medium at 10 μg/ml concentration and observed after 24 and 48 hours. The MIT assay was carried out to check the viability of the cells. FIG. 5 shows the cytotoxic effect of different fractions in the human hepatoma Huh-7 cells and FIG. 6 shows the cytotoxic effect of different fractions in monkey kidney cell line Cos-1. The methanol extract and its fractions 8 and 9 exhibited strong cytotoxic activity in both the cell lines in a dose dependent manner (FIG. 7).

1-5. (canceled)

6. A process for preparing an extract of latex of Calotropis procera for use as cytotoxic and anticancer agents for the prevention and treatment of cancer, comprising:
subjecting a methanol extract of latex of Calotropis procera to the step of solvent extraction using polar and non-polar solvents, wherein the step comprises a first step of extraction with petroleum ether followed by extraction with petroleum ether and chloroform, and followed by a plurality of steps of fractionation with chloroform and methanol.

7. The process as claimed in claim 6, wherein the step of solvent extraction is performed using a non-polar solvent followed by a polar solvent.

8. The process as claimed in claim 6, wherein the step of solvent extraction with non-polar solvents comprises extraction with decreasing amounts of petroleum ether.

9. The process as claimed in claim 6, wherein the step of solvent extraction with non-polar solvent comprises a plurality of steps with increasing amounts of methanol.

10. An aqueous suspension of latex of Calotropis procera produced according to the process of claim 6.
